(19) United States
(12) Patent Application Publication
(10) Pub. No.: US 2006/0235349 A1
(43) Pub. Date: Oct. 19, 2006

(54) Implantable anti-clogging device for maintenance of cerebrospinal fluid shunt patency

(76) Inventors: Brett Osborn, Aventura, FL (US); Simeon Jaggernauth, Tulsa, OK (US)

Correspondence Address: PAUL D. BIANCO: FLEIT, KAIN, GIBBONS,;GUTMAN, BONGINI, & BIANCO P.L., 21355 EAST DIXIE HIGHWAY, SUITE 115, MIAMI, FL, 33180, US

(21) Appl. No.: 11/403,318

(22) Filed: Apr. 13, 2006

Related U.S. Application Data
(60) Provisional application No. 60/594,517, filed on Apr. 14, 2005.
(60) Provisional application No. 60/596,196, filed on Sep. 7, 2005.

(51) Int. Cl. A61M 5/00; A61M 25/00; A61M 5/32

(52) U.S. Cl. 604/9; 604/266

(57) ABSTRACT

Cerebrospinal fluid shunts (implantable devices for diversion of excess fluid from the brain to other body cavities) used to treat hydrocephalus often malfunction. A common etiology of shunt malfunction is obstruction of the distal catheter tip by accumulating particulate matter such as fat or proteinaceous debris. The proposed implantable device maintains the patency of the cerebrospinal fluid shunt with mechanical energy which serves to “scrub” the catheter lumen of particulate debris. The proposed device accomplishes this by housing a source of mechanical energy which is coupled to the external aspect of the catheter, itself traversing through a bore in the device. The energy source secondarily induces a waveform in the cerebrospinal fluid flowing through the catheter. The fluid waveform exerts shearing forces on the catheter wall and serves to disrupt the formation and accumulation of debris that potentially could occlude the shunt catheter, thereby maintaining patency of the shunt.

### CROSS REFERENCE TO RELATED APPLICATIONS

This application claims the benefit under 35 U.S.C § 119(e) of U.S. Provisional Patent Application No. 60/594,517, filed Apr. 14, 2005, and of U.S. Provisional Patent Application No. 60/596,196, filed Sep. 7, 2005. The entire content of each of these applications is incorporated herein by reference.

### FIELD OF THE INVENTION

The present invention relates to a shunting system, and more particularly, to an implantable anti-clogging device used to maintain cerebrospinal fluid shunt patency.

### BACKGROUND OF THE INVENTION

Hydrocephalus is the pathologic accumulation of cerebrospinal fluid (CSF) in the brain. The disease entity has a variety of clinical manifestations ranging from the more benign triad of normal pressure hydrocephalus (gait ataxia, dementia and urinary incontinence) to those secondary to elevated intracranial pressure. The latter may follow a more malignant course and in fact, prove fatal. Hydrocephalus is treated by diverting excess cerebrospinal fluid from the brain to an alternate body cavity. Most commonly, this is the peritoneal cavity (abdominal compartment). The diversion of fluid is accomplished by a shunting device (described in the prior art) which is surgically implanted into the patient. The proximal limb of the shunt system is introduced into the fluid cavities of the brain (“ventricles”) and the distal end into the peritoneal cavity. Through small apertures, excess CSF can enter the shunt (intracranially) and drain into the peritoneal cavity. There exists an interposed valve which broadly serves to regulate CSF flow through the system.

Cerebrospinal fluid shunts, commonly molded from silicone tubing, are susceptible to failure. In fact, 30-40% of cerebrospinal shunts malfunction after primary placement in the adult population. Such malfunctions, while often benign, may prove dangerous or, if left untreated, fatal. Therefore, the maintenance of cerebrospinal fluid shunt patency is essential to patient wellness. Presently, patients with a malfunctioning shunt undergo revision surgery to reestablish flow through the occluded shunt system. In the adult population, this often requires removal of the shunt catheter from the abdominal compartment, resecting the distal 3-4 cm of the catheter and relocating the catheter within the abdomen. The procedure and the inpatient hospitalization have associated risks and morbidity.

The majority of shunt malfunctions in the adult population are the direct result of an occlusion at the distal shunt catheter tip (outlet). The distal catheter tip, often located in the peritoneal cavity, is susceptible to blockage by proteinaceous debris or fat. Prevention of such blockage (i.e., maintenance of CSF shunt patency) is desired and an object of the present invention.

No current implantable device exists to maintain CSF flow in the distal limb of a shunt catheter. U.S. Pat. No. 5,405,316 describes a cerebrospinal fluid shunt that includes plural openings (at the distal tip) through which the fluid flows. As disclosed in this patent, the openings are arranged so tissue growth through the openings does not occlude flow of the fluid within the catheter. However, such designs have been associated with high failure rates. U.S. Pat. No. 5,584,314 describes a self-cleaning inlet head for a fluid. This patent describes an in-line device for the ventricular (proximal) catheter that by combining mechanical and hydraulic action, effectively loosens and sweeps away debris within the catheter lumen. The mechanism is based upon a piston which slides within the catheter lumen in direct contact with potentially debris-laden cerebrospinal fluid. Though elaborate in design, such a mechanism, by virtue of its intricate structure and reliance on small mobile components (see, e.g. FIG. 7), may be susceptible to mechanical failure. An external (extraluminal) method is therefore necessary to maintain fluid flow.

The prior art also includes external energy sources that have been utilized for similar rationale. U.S. Pat. Nos. 4,698,058 and 5,061,255 provide a self-cleaning catheter system for use in protracted transfer of fluid between a patient's body and a point outside the body. The system includes a catheter and a source of mechanical vibration which serves to disintegrate undesirable substances within the catheter lumen and maintain the protracted fluid transfer relatively free from obstruction and contamination by the undesirable substance. Similarly, various forms of energy have been utilized to maintain patency of coronary stents and endovascular grafts (U.S. Pat. Nos. 6,231,516; 6,361,554; and 6,585,763) and to prevent post-interventional neointimal hyperplasia and resultant restenosis (U.S. Pat. Nos. 6,210,393 and 6,755,853).

Other prior art devices which purportedly serve or include methods of utilizing energy to treat or maintain flow of a fluid include those described in U.S. Pat. Nos. 4,509,947; 5,735,811; 5,957,882; and 6,852,097.

Although the prior art has included numerous devices utilizing externally applied energy to maintain flow of fluid in a lumen, there remains a need in the art for a discrete implantable device used to maintain flow of fluid in the distal cerebrospinal fluid shunt limb. This should be differentiated from a shunt system which, as an integrated component, includes an anti-clogging agent. Failure of integrated components in such unitized systems may necessitate replacement of the entire shunt system (both cranial and abdominal limbs), exposing the patient to added surgical risk. It is optimal therefore to utilize a discrete device with an integrated energy source that is surgically implanted in juxtaposition to the distal shunt catheter. A device distinct from the shunt catheter, the proposed invention may be implanted at time of initial shunt placement or during distal catheter revision procedure.

It is an object of the invention to provide an implantable device that maintains cerebrospinal fluid shunt patency.

It is a further object of the invention to provide a device that is implanted in-line with a cerebrospinal fluid shunt.

It is another object of the invention to provide an implantable device that supplies pulsatile energy to the external aspect of a shunt catheter.

It is yet another object of the invention to provide an implantable device capable of inducing a waveform in the cerebrospinal fluid flowing within a shunt catheter.

### SUMMARY OF THE INVENTION

In accordance with the present invention, there is provided an implantable device that maintains cerebrospinal fluid shunt patency. The device includes a housing made of a biocompatible material and enclosing a compartment therein. The housing has a passageway therethrough with entry and exit ports. The lumen of the passageway is configured and dimensioned to receive a section of the shunt tube. The device also includes an energy generator contained within the compartment. The energy generator generates vibratory energy transmitted to the section of the shunt tube to thereby maintain fluid flow in the shunt.

The energy generator can include a transducer that converts electrical energy into the vibratory energy. In one embodiment, the transducer produces ultrasonic vibratory energy. In other embodiments, other forms of energy such as sonic, subsonic, and electromagnetic radiation (e.g. light) are used. An impedance-matched elastic membrane can couple the transducer to the section of the shunt tube. The energy generator can also include a signal generator connected to the transducer for generating an electrical signal, a control unit connected to the signal generator for controlling the electrical signal, and a power source for powering the control module.

In one embodiment, the power source is a rechargeable battery and the energy generator further includes an induction loop connected to the rechargeable battery and responsive to an external energy source to charge the rechargeable battery.

In order to provide a snug connection, each of the entry and exit ports can include a cuff configured and dimensioned to establish contact areas with the section of the shunt tube. The housing can include fixation tabs for attaching the device to tissue.

The present invention also relates to a method of maintaining patency of a shunt implanted in a patient. The anti-clogging device is implanted in the patient such that a section of shunt tube is received in the passageway of the housing. The section of the shunt located within the device is excited with energy generated by the energy generator. This secondarily induces a waveform in the fluid flowing within the shunt tube lumen. The fluid waveform propagates the length of the shunt tube and mechanically impedes the accumulation of proteinaceous debris or fat within the lumen, thereby maintaining patency of the shunt.

The energy generated by the energy generator can be ultrasonic vibratory energy that is generated by a transducer that converts electrical energy into the ultrasonic vibratory energy. In other embodiments, different forms of energy such as sonic, subsonic, and electromagnetic radiation (e.g. light) are generated by the transducer. Regardless of the type of energy, the electrical energy can be stored by a power source, such as a rechargeable battery, that is implanted as a component of the anti-clogging device. The rechargeable battery can be recharged by an extra-corporeal energy source.

The anti-clogging device can be implanted in the patient during the same procedure that the shunt is implanted. Alternatively, the anti-clogging device is implanted in the patient in a procedure subsequent to the procedure that the shunt is implanted.

If the shunt is a cerebrospinal fluid shunt, the section of the tube contained within the housing can be located in the peritoneal cavity of the patient.

### BRIEF DESCRIPTION OF THE DRAWINGS

A complete understanding of the present invention may be obtained by reference to the accompanying drawings, when considered in conjunction with the subsequent detailed description in which:

FIG. 1 is a top detail view of the implantable device in accordance with the invention;

FIG. 2 is a top partial view of the implantable device in accordance with the invention;

FIG. 3 is an electronic schematic of an embodiment of an electrical circuit for the implantable device in accordance with the invention;

FIG. 3A is a schematic representation of an electrical signal input to the implantable device and the resulting mechanical signal output;

FIG. 4 is a sectional view of the implantable device in accordance with the invention;

FIG. 5 is a front sectional view of the implantable device in accordance with the invention;

FIG. 6 is an in situ view of the implantable device in accordance with the invention; and

FIG. 7 is a sectional view of a prior art shunt head that can be used with the implantable device in accordance with the invention.

For purposes of clarity and brevity, like elements and components will bear the same designations and numbering throughout the Figures.

### DETAILED DESCRIPTION OF THE INVENTION

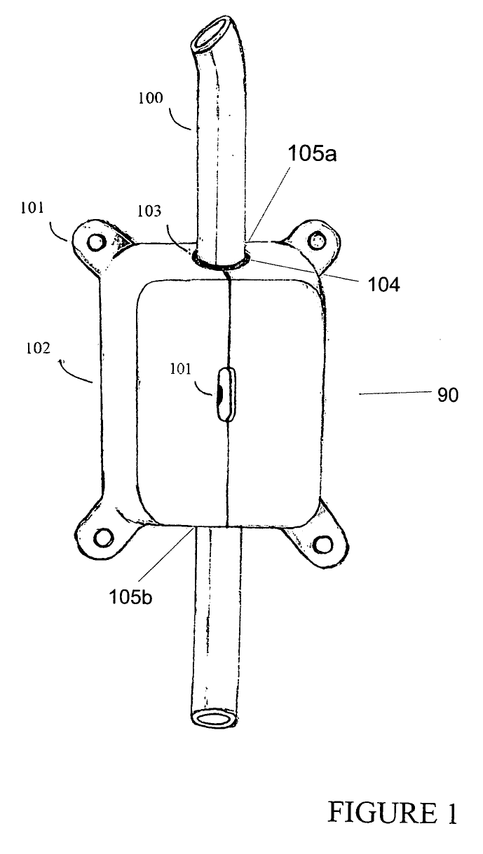

FIG. 1 shows an anti-clogging device 90 of the present invention. Anti-clogging device 90 maintains patency of tube 100 of a shunt. Anti-clogging device 90 includes a housing 102 that may be composed of titanium or any other suitable biocompatible material. Housing 102 provides a protective outer shell for the implantable device and has a hollow core thereby defining a compartment 92 to accommodate the device components. Housing 102 can be provided with smooth or otherwise blunted edges to minimize tissue trauma and may be variously shaped in alternate embodiments. In an exemplary embodiment, housing 102 has a flat bottom surface to maximize contact with the tissue to which it is secured. Shunt catheter tube 100 is traversing device 90 through central bore or passageway 103 which has a lumen configured and dimensioned to receive a section of tube 100.

Passageway 103 can be cuffed with a rubber bushing 104 (at points of entry 105a and exit 105b ports) to encourage tight coupling between tube 100 and the inner lumen of passageway 103. Tube 100 is fed through passageway 103 and subsequently into the chosen body cavity at time of implantation. Alternatively, housing 102 can be provided with a door or other opening so that tube 100 can be inserted or removed without having to be fed through entry 105a and exit 105b ports. As shown, suture tabs 101 are firmly affixed to the corners and top surface of housing 102, but placement may be varied in alternate embodiments. In an exemplary embodiment, suture tab 101 is comprised of flexible material (e.g. silicone) with a central hole that allows for surgical fixation of the device to local tissue at time of implantation.

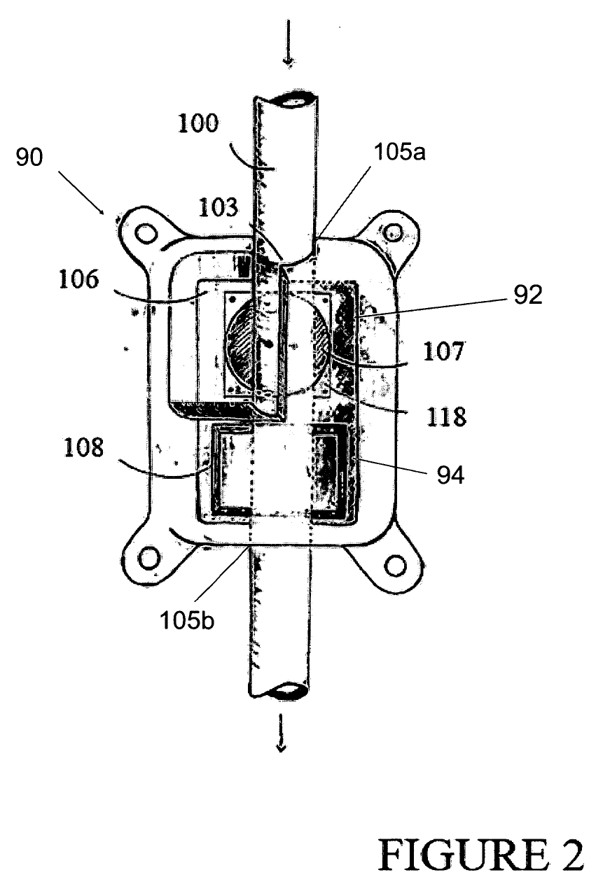

FIG. 2 is a top partial view of implantable anti-clogging device 90 in accordance with the invention. Shunt catheter 100 is traversing device 90 through passageway 103 en route to its final destination in the selected body cavity. Device 90 has both entry 105a and exit 105b ports for traversing shunt catheter 100. Shunt catheter 100, by virtue of its position in passageway 103, is juxtaposed to elastic membrane 118. Elastic membrane 118, which may be composed of any deformable substance (e.g. rubber) is tightly coupled to the active surface of transducer 107. “Active surface” herein refers to the portion of transducer 107 which emits various energy forms. Elastic membrane 118 can be transparent or translucent for visualization of the underlying transducer 107 and can be impedance-matched to the vibrations produced by transducer 107.

Transducer 107 is a component of energy generator 94 that generates vibratory energy transmitted to the section of tube 100 that traverses through device 90 to thereby maintain fluid flow in the shunt. Energy generator 94 is completely contained within compartment 92, and as described in more detail below, can include other components.

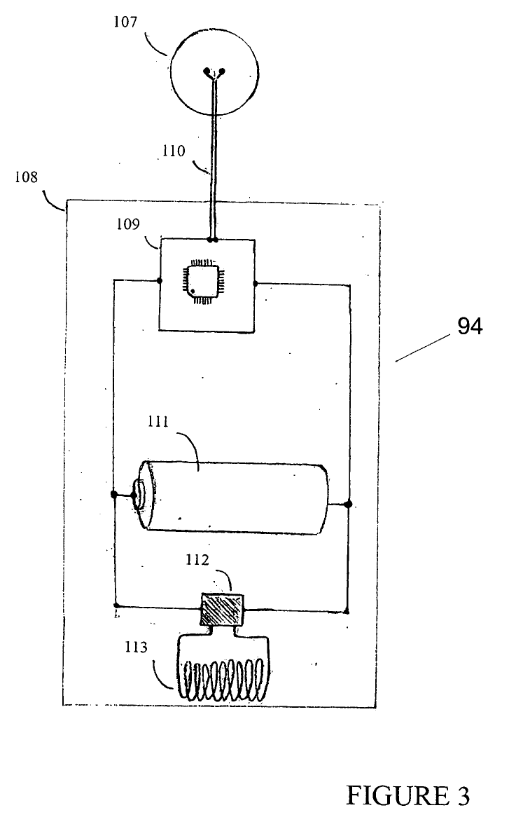

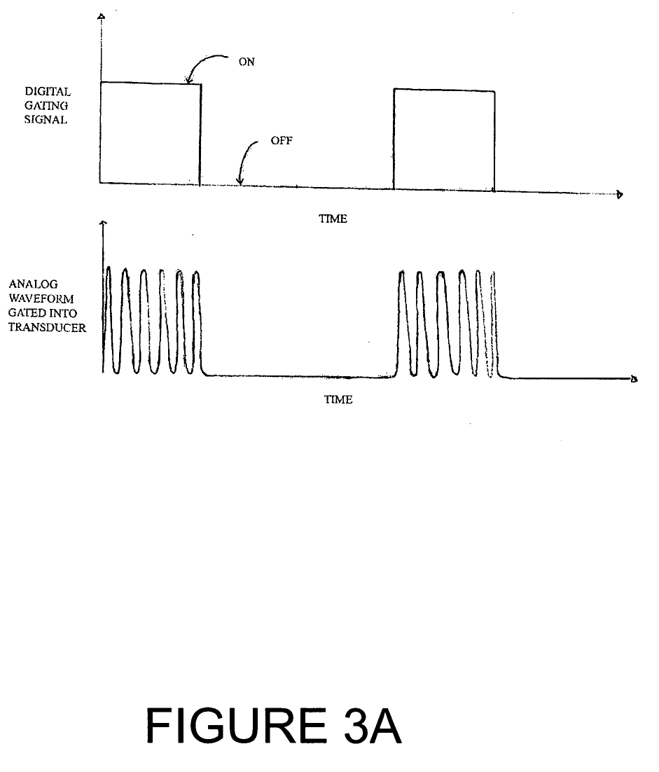

For example and as shown in FIGS. 2 and 3, energy generator 94 can include a circuit board 106 that serves a dual purpose. It functions as a base structure to which the internal components of device 90 are mounted. Circuit board 106 can be made of coated copper or other suitable conductive material such as gold. Energy generator 94 can also include a control unit or module 108 that has wiring etched into circuit board 106. Surface-mount electronic components are utilized in an exemplary embodiment. Alternate embodiments may employ different wiring substrates and components. Coupling leads 110 transmit generated signals from control module 108 to transducer 107.

Control module 108 can include a signal generator 109, a charging unit 112, a rechargeable battery 111, and an induction loop 113. Signal generator 109 is microprocessor-based and comprises an oscillator, gating circuitry, and an amplifier. Control module 108, a functional unit and not a discrete component, generates the amplified waveforms that will ultimately be gated or directed into transducer 107 via coupling leads 110. Transducer 107 which may be sonic, ultrasonic or vibrational emits energy upon its activation and secondarily induces a similar waveform in elastic membrane 118. Coupled to traversing shunt catheter 100, elastic membrane 118 evokes yet a similar waveform in the cerebrospinal fluid flowing within the catheter lumen. Alternate embodiments may utilize electromagnetic radiation and a varied signal transduction scheme to apply light energy to the external aspect of shunt catheter 100.

Charging unit 112 governs the charging of rechargeable battery 111. The circuitry regulates the transfer of energy from induction loop 113 to rechargeable battery 111. In an embodiment, rechargeable battery 111 is charged by exposing inductive loop 113 to an extracorporeal magnetic field source. In an alternate embodiment, rechargeable battery 111 may be substituted for a fuel cell.

FIG. 3 is an electronic schematic view of energy generator 94. Specifically, the basic interconnections of control module 108 are depicted. Rechargeable battery 111 which is the power source is connected in parallel to charging unit 112 and signal generator 109. Induction loop 113 is similarly wired in parallel to charging unit 112. Conducting the waveform from signal generator 109 to transducer 107 are coupling leads 110.

Signal generator 109 comprises an oscillator, gating circuitry and an analog amplifier. The oscillator is capable of generating various waveforms. Generated analog waveforms are gated into transducer 107 at various intervals. The gating circuitry, functionally a square wave generator in series with a digital switch, controls the interval between analog pulses and the pulse width of the analog waveform gated into transducer 107. The former is a function of the duty cycle of the gating pulse and the latter is a function of the frequency of the gating pulse. The frequency of the analog waveform gated into transducer 107 may be varied. In an exemplary embodiment an ultrasonic transducer 107 is utilized, and accordingly analog waveforms with ultrasonic frequencies are gated into transducer 107. Alternate embodiments may utilize sonic or subsonic transducers and driving frequencies. Yet other embodiments may utilize vibrational transducers and appropriate driving frequencies.

FIG. 3A depicts an analog waveform that is gated into transducer 107 by the digital gating pulse. The amplified waveform is gated into transducer 107 via coupling leads 110 at a certain duty cycle and frequency.

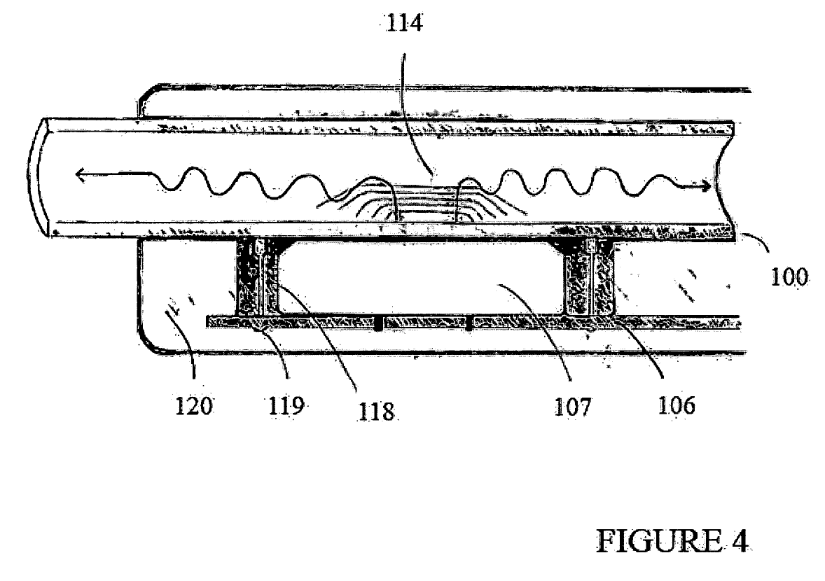

FIG. 4 is a sectional view of device along the longitudinal axis and parallel to the traversing shunt catheter 100. The base of transducer 107 is affixed to the supporting circuit board 106. Elastic membrane 118 is coupled to the active surface of transducer 107. Elastic membrane 118 is positioned in juxtaposition to traversing shunt catheter 100. Fluid waveform 114 within shunt catheter 100 is induced by energy transfer from elastic membrane 118. Retaining screw 119 secures elastic membrane 118 to circuit board 106. Circuit board 106 is encapsulated in epoxy 120 or similar substrate and affixed to the device housing 102 with mounting hardware.

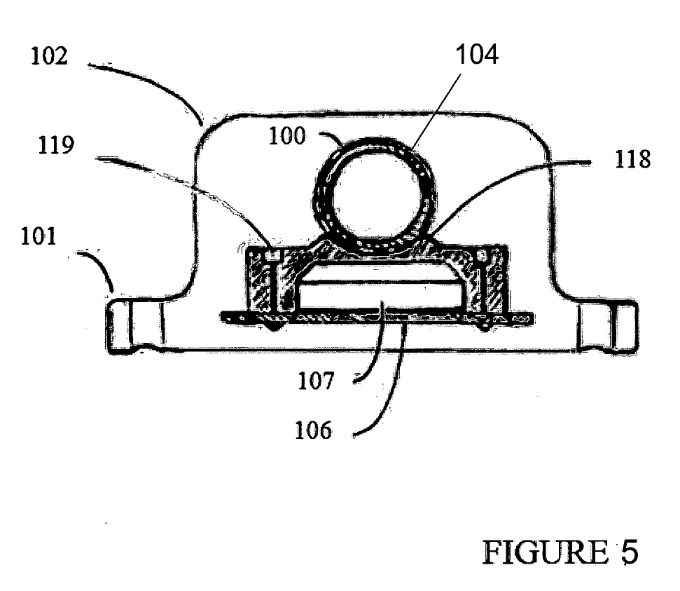

FIG. 5 is a sectional view of device 90 perpendicular to traversing shunt catheter 100 in the vertical plane.

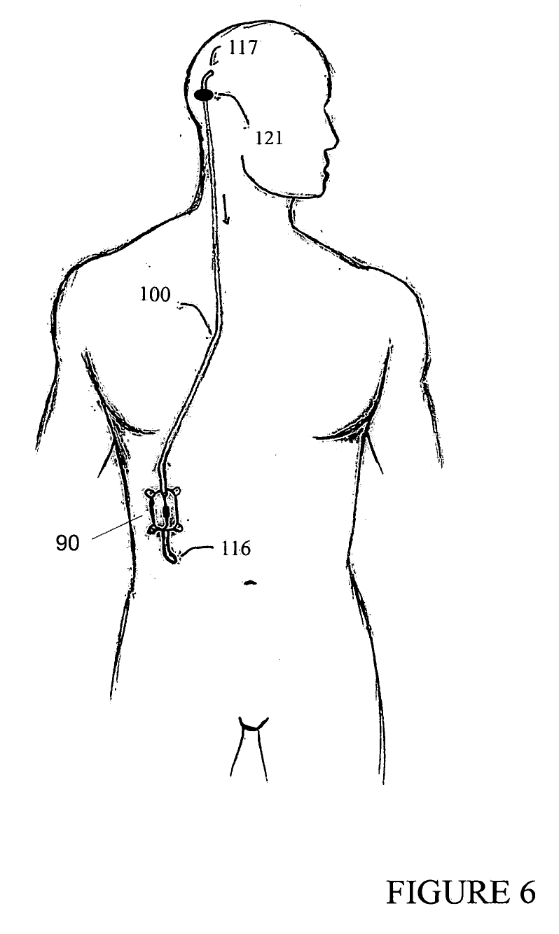

FIG. 6 is an in situ view of device 90. As shown, device 90 is affixed to locally available abdominal fascia. Alternate embodiments of smaller dimension may be implanted in the thoracic region in the case of ventriculopleural or ventriculoatrial shunts. Proximal portion 117 of shunt 100 is located in the ventricular system of the brain. Cerebrospinal fluid enters proximal portion 117 through orifices at its tip. The fluid flows through intervening shunt valve 121 into distal portion 116 of shunt 100. Distal portion 116 is the subcutaneous catheter segment distal to shunt valve 121. Distal shunt catheter 116 prior to its termination in the peritoneal cavity traverses device 90 via passageway 103. Within passageway 103, the external aspect of the catheter is exposed to an energy source which induces a waveform in the flowing cerebrospinal fluid.

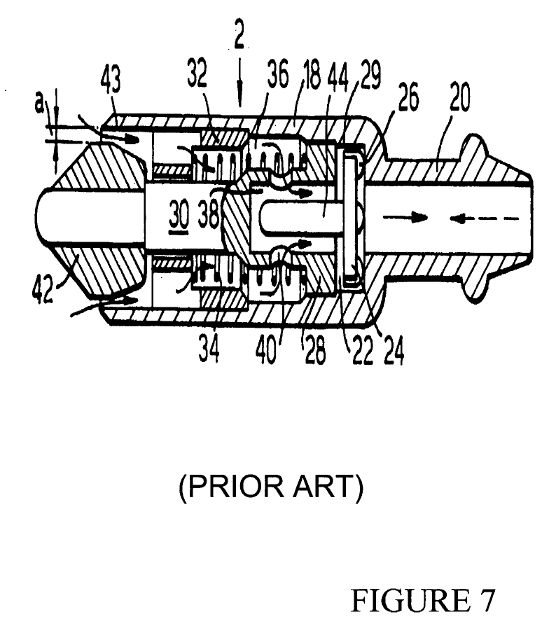

FIG. 7 is a section view of a prior head that can be used on shunt 100 in conjunction with device 90.

Operation

Device 90 maintains the patency of the distal shunt catheter 116 and may be implanted during initial shunt placement or during revision surgery. In the case of a vertriculoperitoneal shunt, the surgeon positions device 90 in-line with the distal shunt catheter 116 prior to its passage into the peritoneal cavity. The surgeon has the option of coupling the catheter with the device before or after the laparotomy is performed. “Laparotomy” refers to the act of surgically entering the peritoneal cavity. Regardless of the selected method, final implantation of the device entails inserting the catheter through passageway 103 and subsequently into the peritoneal cavity.

As optimal function of device 90 relies upon tight coupling between the external aspect of distal shunt catheter 116 and internal elastic membrane 118 for adequate energy transfer, the distal catheter must be pulled through passageway 103 as it is of smaller internal diameter than the external diameter of distal shunt catheter 116. Initially a suture can be passed through passageway 103 and tied to the end of distal catheter 116. The catheter 100 is then pulled through passageway 103 utilizing the passed suture. In another embodiment and as previously discussed, device 90 can be made so that passageway 103 is accessible without having to feed catheter 100. For example, device 90 made be provided in two halves that allow catheter 100 to be placed in passageway 103. The catheter is then directed into the peritoneal. The standard closing procedure involves re-approximating all anatomical layers, deepest to most superficial. Upon closure of the fascia deep to device 90, the surgeon secures device 90 to it utilizing suture tab 101. This prevents subcutaneous migration of the device.

The device requires intermittent recharging (or replacement of the power source) which can be performed with an extracorporeal magnetic field generator. This is placed over the implantation site for a determined time interval.

Advantages

From the description above, a number of advantages of the proposed invention become evident:
(a) By reducing the incidence of shunt malfunctions, the proposed device reduces the number of revision procedures necessary. This translates to a potential reduction in surgical morbidity (as an absolute number) and cost.
(b) Such a device is easily implanted either at time of initial surgery or during revision procedures.
(c) The proposed device may be placed in-line with existing shunt catheters in the case of distal revision surgery during which the catheter is removed from the peritoneal cavity and relocated to another site within the same cavity.
(d) As the proposed device is externally coupled to the shunt catheter 100, there are no intraluminal components capable of causing a shunt malfunction.
(e) Similarly, the proposed device is, in itself, not subject to malfunction as caused by accumulation of proteinaceous debris and/or fat within the distal catheter lumen.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention. In addition, unless mention was made above to the contrary, it should be noted that all of the accompanying drawings are not to scale. All references identified are incorporated herein by reference.

What is claimed is:

1. An implantable anti-clogging device for maintaining patency of a shunt that includes a tube, the device comprising:
a housing made of a biocompatible material, enclosing a compartment therein, and having a passageway therethrough with entry and exit ports, the passageway having a lumen configured and dimensioned to receive a section of the shunt tube; and
an energy generator contained within the compartment, the energy generator generating vibratory energy transmitted to the section of the shunt tube to thereby maintain fluid flow in the shunt.

2. The implantable anti-clogging device of claim 1 wherein the energy generator includes a transducer that converts electrical energy into the vibratory energy.

3. The implantable anti-clogging device of claim 2 wherein an impedance-matched elastic membrane couples the transducer to the section of the shunt tube.

4. The implantable anti-clogging device of claim 3 wherein the energy generator further includes:
a signal generator connected to the transducer for generating an electrical signal;
a control unit connected to the signal generator for controlling the electrical signal; and
a power source for powering the control module.

5. The implantable anti-clogging device of claim 4 wherein the transducer produces ultrasonic vibratory energy.

6. The implantable anti-clogging device of claim 5 wherein the power source is a rechargeable battery.

7. The implantable anti-clogging device of claim 6 wherein the energy generator further includes an induction loop connected to the rechargeable battery and responsive to an external energy source to charge the rechargeable battery.

8. The implantable anti-clogging device of claim 7 wherein each of the entry and exit ports includes a cuff configured and dimensioned to establish contact areas with the section of the shunt tube.

9. The implantable anti-clogging device of claim 4 wherein the housing includes fixation tabs for attaching the device to tissue.

10. A method of maintaining patency of a shunt implanted in a patient, the method comprising the steps of:
implanting the anti-clogging device of claim 1 in the patient such that a section of shunt tube is received in the passageway of the housing; and
inducing vibration in the section of the shunt tubing with the vibratory energy generated by the energy generator.

11. The method of claim 10 wherein the energy generator includes a transducer that converts electrical energy into the vibratory energy.

12. The method of claim 11 wherein the electrical energy is stored by a power source implanted as a component of the anti-clogging device.

13. The method of claim 12 wherein the vibratory energy is one of ultrasonic, subsonic, or sonic vibratory energy.

14. The method of claim 13 wherein the vibratory energy is ultrasonic vibratory energy.

15. The method of claim 13 wherein the power source is a rechargeable battery.

16. The method of claim 15 wherein the rechargeable battery is recharged by an extra-corporeal energy source.

17. The method of claim 12 wherein the anti-clogging device is implanted in the patient during the same procedure that the shunt is implanted.

18. The method of claim 12 wherein the anti-clogging device is implanted in the patient in a procedure subsequent to the procedure that the shunt is implanted.

19. The method of claim 12 wherein the shunt is a cerebrospinal fluid shunt.

20. The method of claim 19 wherein the section of the tube is located in the peritoneal cavity of the patient.
